(19) United States
(12) Patent Application Publication
(10) Pub. No.: US 2023/0011235 A1
(43) Pub. Date: Jan. 12, 2023

(54) Dielectric Spectroscopic Measurement Device

(71) Applicant: Nippon Telegraph and Telephone Corporation, Tokyo (JP)

(72) Inventors: Michiko Seyama, Tokyo (JP); Masahito Nakamura, Tokyo (JP)

(21) Appl. No.: 17/783,244

(22) PCT Filed: Dec. 16, 2019
(86) PCT No.: PCT/JP2019/049169

§ 371 Date: Jun. 7, 2022

(51) Int. Cl. G01N 22/00; G01N 27/02

(52) U.S. Cl. CPC G01N 22/00; G01N 27/026

(57) ABSTRACT

A dielectric spectrometer includes an apparatus main body including a dielectric and in which a flow channel is formed and a probe including a high frequency line. The probe measures a dielectric constant of an object substance as an electrical signal and penetrates the apparatus main body, one end that is an open end of the probe functions as a detection terminal that is exposed to inside the flow channel, and a fringe is formed at the detection terminal of the probe.

This patent application is a national phase filing under section ₃₇1 of PCT application no. PCT/JP2019/049169, filed on Dec. 16, 2019, which application is hereby incorporated herein by reference in its entirety.

### TECHNICAL FIELD

The present invention relates to a dielectric spectrometer for measuring a complex dielectric constant of a minute amount of a liquid sample.

### BACKGROUND

As the population ages, how to respond to adult-onset diseases has become a major challenge. Checking a blood sugar level or the like requires collecting blood and therefore poses a major burden on a patient. Therefore, non-invasive component concentration measuring apparatuses that do not collect blood are attracting attention.

As a non-invasive component concentration measurement, a technique that uses electromagnetic waves in microwave and millimeter-wave bands is proposed. The technique has an advantage over optical measurement such as measurement using near-infrared light in that there is less scatter within an organism, a single photon has less energy, and the like. An example of using electromagnetic waves in the microwave and millimeter-wave bands is a measurement technique using a resonance structure described in NPL 1. In this technique, a measurement device with a high Q value such as an antenna or a resonator and a measurement sample are brought into contact with each other to measure frequency characteristics around a resonance frequency. Since the resonance frequency is determined by a complex dielectric constant around the measurement device, by predicting a correlation between an amount of shift of the resonance frequency and a component concentration, the component concentration is estimated based on the amount of shift of the resonance frequency.

As another measurement technique that uses electromagnetic waves in microwave and millimeter-wave bands, dielectric spectroscopy described in PTL 1 is proposed. The dielectric spectroscopy involves irradiating the inside of skin with an electromagnetic wave, absorbing the electromagnetic wave in accordance with an interaction between a blood component such as glucose molecules being a measurement object and water, and observing an amplitude and a phase of the electromagnetic wave. A dielectric relaxation spectrum is calculated based on the amplitude and the phase with respect to a frequency of the measured electromagnetic wave.

Generally, a dielectric relaxation spectrum is expressed as a linear combination of relaxation curves based on the Cole-Cole equation for calculating a complex dielectric constant. In the measurement of a biogenic substance, a complex dielectric constant is correlated to an amount of a blood component such as glucose or cholesterol that is contained in blood, and the biogenic substance is measured as an electrical signal (amplitude, phase) that corresponds to a change in the complex dielectric constant. A calibration model is created by measuring the correlation between a change in the complex dielectric constant and a component concentration in advance, and the component concentration is calibrated by comparing a measured change in the complex dielectric constant and the calibration model to each other. Whichever measurement technique is to be used, since an improvement in measurement sensitivity can be expected by selecting a frequency band with a strong correlation with a component to be measured, it is important to measure, in advance, a change in the dielectric constant by wide band dielectric spectroscopy.

Among dielectric spectroscopy, a dielectric spectroscopic method using a device in which microchannels are integrated in a coplanar waveguide (CPW) such as that described in NPL 2 enables dielectric constant information in DC-100 GHz bands to be acquired and, since a sample can be measured in an amount of around several 10 microliters, the dielectric spectroscopic method is suitable for dielectric spectroscopic characteristics of expensive and rare substances such as biological samples.

### Citation List

### Patent Literature

[PTL 1] Japanese Patent Application Laid-open No. 2013-032933

### Non Patent Literature

[NPL 1] M. Hofmann et al., “Microwave-Based Noninvasive Concentration Measurements for Biomedical Applications”, IEEE Transactions on Microwave Theory and Techniques, vol. 61, no. 5, pp. 2195-2204, 2013.

[NPL 2] K. Grenier et al., “Integrated Broadband Microwave and Microfluidic Sensor Dedicated to Bioengineering”, IEEE Transactions on Microwave Theory and Techniques, vol. 57, no. 12, pp. 3246-3253, 2009.

### SUMMARY

### Technical Problem

However, conventional dielectric spectrometers have the following problem. In a dielectric spectrometer of this type, microchannels are integrated in a transmission line that can be fabricated using a printed substrate such as a microstrip line or a coplanar waveguide. As such, in a conventional dielectric spectrometer, microchannels are installed on a propagation path of an electromagnetic wave. Therefore, in measurement using a conventional dielectric spectrometer, multiple reflections attributable to a change in characteristic impedance of the transmission line occur and an unnecessary multiple reflection component is superimposed as a measurement error during wide band dielectric constant measurement. In this manner, with a conventional dielectric spectrometer, there is a problem in that wide band dielectric constant measurement cannot be accurately performed.

Embodiments of the present invention have been made in order to solve the problem described above and an object thereof is to enable wide band dielectric constant measurement to be performed in an accurate manner.

### Means for Solving the Problem

A dielectric spectrometer according to embodiments of the present invention includes: an apparatus main body in which a flow channel is formed and which is constituted of a dielectric; and a probe which is constituted of a high frequency line, which penetrates the apparatus main body, and of which one end that is an open end functions as a detection terminal that is exposed to inside the flow channel, wherein a guiding direction of the high frequency line is perpendicular to a flow of the flow channel, and in the probe, a fringe is formed at the detection terminal.

### Effects of Embodiments of the Invention

As described above, according to embodiments of the present invention, since a fringe is formed at a detection terminal of a probe constituted of a high frequency line, wide band dielectric constant measurement can be performed in an accurate manner.

### BRIEF DESCRIPTION OF THE DRAWINGS

FIG. 1A is a sectional view showing a configuration of a dielectric spectrometer according to an embodiment of the present invention.

FIG. 1B is a plan view showing a configuration of the dielectric spectrometer according to an embodiment of the present invention.

FIG. 2 is a configuration diagram showing a configuration of a measurement system using the dielectric spectrometer according to an embodiment of the present invention.

FIG. 3 is a characteristic diagram showing a result of a simulation of an attenuation rate of electric field intensity by a probe.

FIG. 4 is a characteristic diagram showing a result of a simulation of an attenuation rate in a guiding direction from a detection terminal of electric field intensity by a probe.

FIG. 5 is a characteristic diagram showing a result of analyzing an S parameter when air is an object substance of the dielectric spectrometer according to an embodiment.

FIG. 6 is a characteristic diagram showing a result of analyzing an S parameter when air is an object substance of a conventional dielectric spectrometer.

FIG. 7 is a configuration diagram showing a configuration of the dielectric spectrometer according to an embodiment of the present invention.

### DETAILED DESCRIPTION OF ILLUSTRATIVE EMBODIMENTS

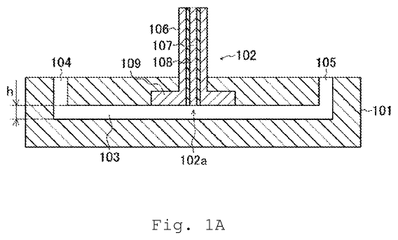

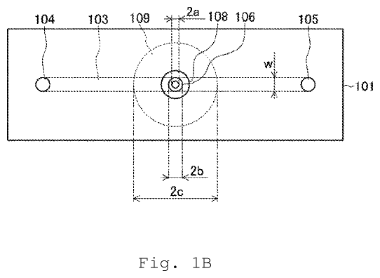

Hereinafter, a dielectric spectrometer according to an embodiment of the present invention will be described with reference to FIGS. 1A and 1B. The dielectric spectrometer includes an apparatus main body 101 constituted of a dielectric and a probe 102 constituted of a high frequency line. A flow channel 103 is formed in the apparatus main body 101. An object substance to be a measurement object flows through the flow channel 103. An intake 104 and an outlet 105 are also formed in the apparatus main body 101 so as to connect to the flow channel 103. For example, the apparatus main body 101 can be constituted of a resin such as polydimethylsiloxane (PDMS) or polymethyl methacrylate (PMMA). For example, an external shape of the apparatus main body 101 is a cuboid.

In addition, the probe 102 measures a dielectric constant of the object substance as an electrical signal and penetrates the apparatus main body 101, and one end that is an open end of the probe 102 functions as the detection terminal 102a that is exposed to inside a flow channel 103. Furthermore, in the probe 102, a guiding direction of the high frequency line is perpendicular to a flow of the flow channel 103. Moreover, in the dielectric spectrometer according to the embodiment, a fringe 109 is formed at the detection terminal 102a of the probe 102. In this example, the fringe 109 with a disk shape is provided at an end of a main body of the probe 102 with a columnar shape. For example, the probe 102 is constituted of a coaxial line including an outer conductor 106 and an inner conductor 107 and the fringe 109 is formed on the outer conductor 106. A space between the outer conductor 106 and the inner conductor 107 is filled by a dielectric layer 108 constituted of a fluororesin or the like.

The probe 102 is used to measure an electric characteristic such as impedance, admittance, or a complex dielectric constant of a sample being a measurement object by utilizing a leakage electromagnetic field that is created between the outer conductor 106 and the inner conductor 107 which come into contact with the object substance (fluid) inside the flow channel 103 at the detection terminal 102a.

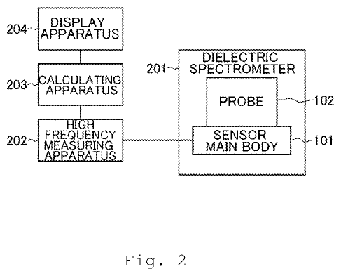

A dielectric spectroscopic system will now be described with reference to FIG. 2. The system includes a dielectric spectrometer 201 according to embodiments of the present invention, a high frequency measuring apparatus 202, a calculating apparatus 203, and a display apparatus 204. The dielectric spectrometer 201 includes the apparatus main body 101 and the probe 102 described above. The high frequency measuring apparatus 202 sweeps frequencies within an arbitrary range to generate an electromagnetic wave and supplies the probe 102 with the electromagnetic wave. In addition, the high frequency measuring apparatus 202 measures (observes) an amplitude and a phase of the electromagnetic wave in a state where the electromagnetic wave is absorbed by the object substance in the probe 102. The calculating apparatus 203 calculates a dielectric constant of the object substance from a result of a measurement performed by the high frequency measuring apparatus 202. The display apparatus displays a result of a calculation performed by the calculating apparatus 203.

For example, the high frequency measuring apparatus 202 is a vector network analyzer. In addition, a commercially available impedance analyzer, an LCR meter, or the like can be used as the high frequency measuring apparatus 202. Furthermore, a dielectric spectroscopic system can also be constructed based on an S parameter measurement system using an arbitrary waveform generator and a wideband measuring instrument or based on an impedance measurement system using a bridge method or an RF-IV method.

Next, the dielectric spectrometer according to an embodiment will be described in greater detail. A characteristic impedance of the probe 102 constituted of a coaxial line is expressed by Equation (1) below. In Equation (1), Z0 denotes a characteristic impedance (Ω) of a coaxial line, Εᵣ denotes a relative dielectric constant of the dielectric layer 108 in the coaxial line, a denotes a radius of an outer diameter of the inner conductor 107, and b denotes a radius of an inner diameter of the outer conductor 106. In addition, a cutoff frequency of the coaxial line is expressed by Equation (2) below. In Equation (2), f_{c} denotes a cutoff frequency and v denotes the speed of light.

$$Equations ( 1 ) and ( 2 )$$

$$Z 0 = \frac{138.061}{\sqrt{{ε}_{r}}} log \frac{b}{a} \qquad ( 1 )$$

$$fc = \frac{v}{π \sqrt{{ε}_{r}} ( a + b )} \qquad ( 2 )$$

For example, the high frequency measuring apparatus 202 constituted of a vector network analyzer is generally designed so as to have a characteristic impedance of 50 Ω or 75 Ω. Therefore, the parameters a, b, and Εᵣ are designed so that an upper limit of measurement frequencies does not equal or fall below the cutoff frequency f_{c} and, at the same time, the characteristic impedance satisfies the above. For example, when the upper limit of measurement frequencies is 50 GHz, the characteristic impedance is 50 Ω, and the dielectric layer 108 between the outer conductor 106 and the inner conductor 107 is fluororesin (Ε_{r≈}2.2), a is set to 0.175 mm and b is set to 0.8 mm.

c denotes a radius in a plan view of the fringe 109 (a fringe radius) provided in a portion (the detection terminal 102a) that comes into contact with the object substance of the probe 102. The fringe radius c is formed so as to equal or exceed a region in which an electric field intensity of a leakage field from the detection terminal iota of the probe 102 is equal to or lower than 1% of a maximum value. In other words, a surface in a direction perpendicular to the guiding direction of the high frequency line (the coaxial line) of the fringe 109 is made wider than a region in which an electric field intensity of a leakage field from the detection terminal iota is equal to or lower than 1% of a maximum value. For example, c≥3 mm.

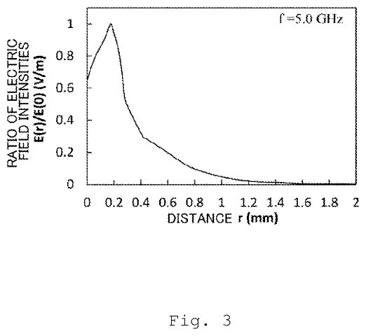

FIG. 3 shows a result of a simulation of an attenuation rate of electric field intensity in a planar direction (a horizontal direction) of the surface of the detection terminal 102a of the probe 102 from a center of the detection terminal 102a when c=9.5 mm. In FIG. 3, r denotes a distance in the horizontal direction from the center of the detection terminal 102a.

In this case, a state is created where there is no gap between a side surface adjacent to the detection terminal 102a of the fringe 109 and a wall surface of the flow channel 103 of the apparatus main body 101 so that the fluid flowing through the flow channel 103 does not leak out. The probe 102 including the fringe 109 is fixed to the apparatus main body 101 using screws, a double-faced tape, or the like. In addition, accuracy of positioning between the apparatus main body 101 and the probe 102 may be increased using a pattern, pins, or the like for positioning. Furthermore, the probe 102 can be configured to prevent leakage of the fluid using an O ring, packing, or the like when the probe 102 is attached to the apparatus main body 101.

A height h of the flow channel 103 in the guiding direction of the high frequency line (the coaxial line) and a width w of the flow channel 103 in a direction perpendicular to the guiding direction of the high frequency line are set within a region in which an electric field intensity of a leakage field from the detection terminal 102a of the probe 102 is equal to or lower than 1% of a maximum value.

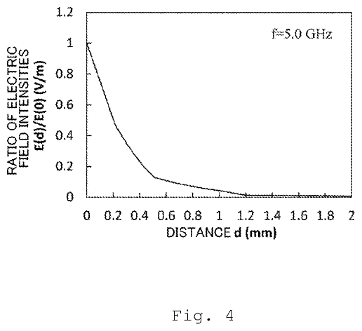

FIG. 4 shows a result of a simulation of an attenuation rate with respect to a distance d in the guiding direction (a height direction of the flow channel 103) from the detection terminal 102a of electric field intensity by the probe 102. The width w and the height h are set equal to or lower than values where the attenuation rate of the electric field intensity is 1% such as h=2 mm and w=3 mm. In regard to the width w, in consideration of a margin for forming the fringe 109, w=fringe radius c-6 (mm) at a maximum.

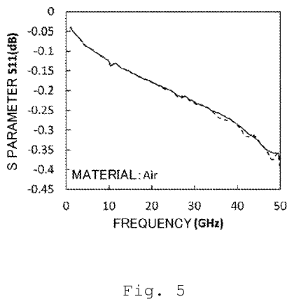

FIG. 5 shows a comparison between cases where the flow channel 103 according to embodiments of the present invention is present and absent. FIG. 5 represents a result of an analysis of an S parameter (scattering parameter) S11 when using the respective parameters described above and when air (dielectric constant=1) is the object substance. A solid line represents a case where the flow channel is absent and a dotted line represents a case where the flow channel is present.

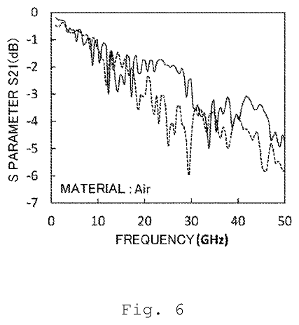

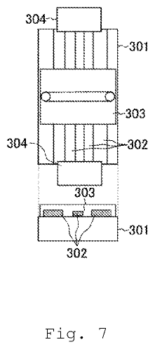

FIG. 6 shows a result of a simulation of an effect of a flow channel in a conventional dielectric spectrometer. In a similar manner to that described above, FIG. 6 represents a comparison between cases where the flow channel is present and absent as well as a result of an analysis of the S parameter S11 when air (dielectric constant=1) is the object substance. A solid line represents a case where the flow channel is absent and a dotted line represents a case where the flow channel is present. As shown in FIG. 7, in the conventional dielectric spectrometer, a wiring 302 that constitutes a coplanar-type high frequency line is formed on a substrate 301 constituted of a dielectric, and a flow channel substrate 303 is formed on the wiring 302. A flow channel is formed so as to be orthogonal to the guiding direction of the high frequency line on the flow channel substrate 303. In addition, as compared to the wiring 302, a high-frequency connector 304 is connected as a resistor.

As shown in FIG. 6, conventionally, since the flow channel substrate that forms a flow channel is installed on a coplanar waveguide which is a propagation path of electric waves, characteristics significantly vary depending on whether the flow channel is present or not. On the other hand, in the result of a simulation shown in FIG. 5, there is hardly any effect of the flow channel. As described above, according to embodiments of the present invention, a measurement of an object substance can be performed in a state where there is hardly any effect of the presence or absence of the flow channel (effect of the apparatus main body 101). In particular, a wide band dielectric constant measurement of an object substance in a minute amount using a microchannel with, for example, a flow channel height of 2 mm and a flow channel width 3 mm can be performed more accurately.

In the measurement of the object substance by the dielectric spectrometer according to the embodiment, a dielectric constant of the object substance is calculated from a measured impedance, admittance, a reflection coefficient, or the like. For example, using three reference substances including a first reference substance, a second reference substance, and a third reference substance of which dielectric constants are known in advance, a sample dielectric constant is calculated using equations (3) and (4) presented below.

$$Equations ( 3 ) and ( 4 )$$

$$\frac{( {ρ}_{4} - {ρ}_{1} ) ( {ρ}_{3} - {ρ}_{2} )}{( {ρ}_{4} - {ρ}_{2} ) ( {ρ}_{1} - {ρ}_{3} )} = \frac{( {y}_{4} - {y}_{1} ) ( {y}_{3} - {y}_{2} )}{( {y}_{4} - {y}_{2} ) ( {y}_{1} - {y}_{3} )} \qquad ( 3 )$$

$${y}_{1} = {ε}_{i} + \frac{{G}_{0}}{j ω {C}_{0}} {ε}_{i}^{5 / 2} \qquad ( 4 )$$

In this case, ρ₁ denotes a reflection coefficient obtained as a result of measuring the first reference substance, ρ₂ denotes a reflection coefficient obtained as a result of measuring the second reference substance, and ρ₃ denotes a reflection coefficient obtained as a result of measuring the third reference. In addition, ρ₄ denotes a reflection coefficient obtained as a result of measuring the object substance.

Furthermore, y₁, denotes a linear map of admittance obtained as a result of measuring the first reference substance with a dielectric constant of Ε₁,y₂ denotes a linear map of admittance obtained as a result of measuring the first reference substance with a dielectric constant of Ε₂, and y₃ denotes a linear map of admittance obtained as a result of measuring the first reference substance with a dielectric constant of Ε₃. In addition, y₄ denotes a linear map of admittance obtained as a result of measuring the object substance with a dielectric constant of Ε₄.

G₀ indicates a characteristic impedance of a portion that is outside of the apparatus main body 101 in the transmission line of the probe 102.

The dielectric constant of the object substance is calculated by using the first reference substance, the second reference substance, and the third reference substance of which dielectric constants are known as calibration standards. As the calibration standards, air, a solid, liquid metal, water, an organic solvent such as alcohol, or the like is used. In the dielectric spectrometer according to the embodiment, since an effect of the flow channel 103 (the apparatus main body 101) with respect to impedance and admittance has been reduced, a conversion to a dielectric constant can be performed while reducing an effect of a reflection term derived from the flow channel during the calculation of Equation (3) or the like. In this manner, using the dielectric spectrometer according to the embodiment enables a dielectric constant of an object in a minute amount in the flow channel 103 to be accurately measured across a wide bandwidth.

As described above, according to embodiments of the present invention, since a fringe is formed at a detection terminal of a probe constituted of a high frequency line, wide band dielectric constant measurement can be performed in an accurate manner.

It is to be understood that the present invention is not limited to the embodiments described above and that many modifications and combinations will obviously occur to those with ordinary skill in the art without departing from the technical scope of the present invention.

### Reference Signs List

101 Apparatus main body

102 Probe

102a Detection terminal

103 Flow channel

104 Intake

105 Outlet

106 Outer conductor

107 Inner conductor

108 Dielectric layer

109 Fringe

1-4. (canceled)

5. A dielectric spectrometer comprising:
an apparatus main body including a flow channel and comprising a dielectric;
a probe comprising a high frequency line having a guiding direction that is perpendicular to a flow of the flow channel, wherein the probe penetrates the apparatus main body, and wherein an open end of the probe is a detection terminal that is exposed to inside the flow channel; and
a fringe disposed at the detection terminal in the probe.

6. The dielectric spectrometer according to claim 5, wherein a surface of the fringe in a direction perpendicular to the guiding direction of the high frequency line is wider than a region in which an electric field intensity of a leakage field from the detection terminal is equal to or lower than 1% of a maximum value.

7. The dielectric spectrometer according to claim 6, wherein a height of the flow channel in the guiding direction of the high frequency line and a width of the flow channel in the direction perpendicular to the guiding direction of the high frequency line are set within the region in which the electric field intensity of the leakage field from the detection terminal is equal to or lower than 1% of the maximum value.

8. The dielectric spectrometer according to claim 7, wherein:
the probe comprises a coaxial line; and
the fringe is disposed on an outer conductor of the coaxial line.

9. The dielectric spectrometer according to claim 5, wherein a height of the flow channel in the guiding direction of the high frequency line and a width of the flow channel in a direction perpendicular to the guiding direction of the high frequency line are set within a region in which an electric field intensity of a leakage field from the detection terminal is equal to or lower than 1% of a maximum value.

10. The dielectric spectrometer according to claim 5, wherein:
the probe comprises a coaxial line; and
the fringe is disposed on an outer conductor of the coaxial line.

11. A method of forming a dielectric spectrometer, the method comprising:
forming an apparatus main body comprising a dielectric;
forming a flow channel in the apparatus main body;
forming a probe comprising a high frequency line, the high frequency line having a guiding direction that is perpendicular to a flow of the flow channel, wherein the probe penetrates the apparatus main body, and wherein an open end of the probe is a detection terminal that is exposed to inside the flow channel; and
forming a fringe at the detection terminal in the probe.

12. The method according to claim ii, wherein a surface of the fringe in a direction perpendicular to the guiding direction of the high frequency line is wider than a region in which an electric field intensity of a leakage field from the detection terminal is equal to or lower than 1% of a maximum value.

13. The method according to claim 12, wherein a height of the flow channel in the guiding direction of the high frequency line and a width of the flow channel in the direction perpendicular to the guiding direction of the high frequency line are set within the region in which the electric field intensity of the leakage field from the detection terminal is equal to or lower than 1% of the maximum value.

14. The method according to claim 13, wherein:
the probe comprises a coaxial line; and
the fringe is formed on an outer conductor of the coaxial line.

15. The method according to claim ii, wherein a height of the flow channel in the guiding direction of the high frequency line and a width of the flow channel in a direction perpendicular to the guiding direction of the high frequency line are set within a region in which an electric field intensity of a leakage field from the detection terminal is equal to or lower than 1% of a maximum value.

16. The method according to claim 11, wherein:
the probe comprises a coaxial line; and
the fringe is disposed on an outer conductor of the coaxial line.
